(19) United States
(12) Patent Application Publication
(10) Pub. No.: US 2022/0110906 A1
(43) Pub. Date: Apr. 14, 2022

(54) ANTIMICROBIAL DENTAL APPLIANCE

(71) Applicant: 3M INNOVATIVE PROPERTIES COMPANY, St. Paul, MN (US)

(72) Inventors: Ta-Hua Yu, Woodbury, MN (US); Timothy J. Hebrink, Scandia, MN (US); Yongshang Lu, Woodbury, MN (US); Karl J.L. Geisler, Saint Paul, MN (US); Narina Y. Stepanova, Woodbury, MN (US); Mary E. Johansen, Woodbury, MN (US); Jay M. Jennen, Forest Lake, MN (US); Bhaskar V. Velamakanni, Woodbury, MN (US); David P. Siglin, St. Paul, MN (US); Mark T. Gibson, Stillwater, MN (US); Troy J. Anderson, Afton, MN (US)

(21) Appl. No.: 17/418,896

(22) PCT Filed: Dec. 30, 2019
(86) PCT No.: PCT/IB2019/061448

§ 371 Date: Jun. 28, 2021

Related U.S. Application Data
(60) Provisional application No. 62/787,027, filed on Dec. 31, 2018.

(51) Int. Cl. A61K 31/23; A61C 7/08; A61K 9/00; A61P 31/04

(52) U.S. Cl. CPC A61K 31/23; A61P 31/04; A61K 9/0053; A61C 7/08

(57) ABSTRACT

A dental appliance includes a polymeric shell with an arrangement of one or more cavities configured to receive one or more teeth, and the polymeric shell includes an antimicrobial lipid, such as monolaurin, and optionally an enhancer.

### BACKGROUND

Orthodontic treatments involve repositioning misaligned teeth and improving bite configurations for improved cosmetic appearance and dental function. Repositioning teeth is accomplished by applying controlled forces to the teeth over an extended time period.

“Braces” include a variety of appliances such as brackets, bands, archwires, ligatures, and O-rings that are bonded to the teeth of a patient. The appliances are periodically replaced or adjusted by an orthodontist to apply the desired forces to the teeth and reposition them to achieve a desired alignment condition.

Teeth may also be repositioned by placing a polymeric incremental position adjustment appliance, generally referred to as an orthodontic aligner or an orthodontic aligner tray, over the teeth of the patient for each treatment stage of an orthodontic treatment. The orthodontic alignment trays include a polymeric shell with a plurality of cavities for receiving one or more teeth. The individual cavities in the polymeric shell are shaped to exert force on one or more teeth to resiliently and incrementally reposition selected teeth or groups of teeth in the upper or lower jaw. A series of orthodontic aligner trays are provided for wear by a patient sequentially and alternatingly during each stage of the orthodontic treatment to gradually reposition teeth from one tooth arrangement to a successive tooth arrangement to achieve a desired tooth alignment condition. Once the desired alignment condition is achieved, an aligner tray, or a series of aligner trays, may be used periodically or continuously in the mouth of the patient to maintain tooth alignment. In addition, orthodontic retainer trays may be used for an extended time period to maintain tooth alignment following the initial orthodontic treatment.

A stage of orthodontic treatment may require that a polymeric orthodontic retainer or aligner tray remain in the mouth of the patient for several hours a day, over an extended time period of days, weeks or even months. While the orthodontic retainer or aligner tray is in use in the mouth of the patient, foods or other substances can stain or otherwise damage the appliance. In addition, microorganisms can contaminate the surface of the appliance, which in some cases can also cause biofilms to form on the surface. The biofilms can be difficult to remove, even if the orthodontic aligner tray is periodically cleaned. Microorganisms or biofilm buildup on the surface of the orthodontic aligner tray can stain or otherwise discolor the aligner tray, can cause undesirable tastes and odors, and even potentially lead to various periodontal diseases.

Antimicrobial articles or coatings have been used to prevent/reduce infections on medical devices such as orthopedic pins, plates and implants, wound dressings, and the like. Metallic ions with antimicrobial properties, such as Ag, Au, Pt, Pd, Ir, Cu, Sn, Sb, Bi, Zn, and the like, have been used as antimicrobial compounds. Various silver salts, complexes and colloids have been used to prevent and control infection on the surfaces of medical devices. The free silver ions in soluble salts of silver can be complexed or removed from a surface and may not provide a sufficiently prolonged release of silver ions to maintain an antimicrobial effect when a dental appliance is used in the mouth of a patient for an extended time period. As a result, soluble silver salts must be reapplied periodically, and reapplication can be burdensome or impractical.

### SUMMARY

In general, the present disclosure is directed to a dental appliance that includes at least one layer with an antimicrobially effective amount of an antimicrobial lipid compound. In some embodiments, an effective amount of an antimicrobial lipid compound or mixture of antimicrobial lipid compound or compounds, such as monolaurin, added to a polymeric material of the dental appliance as a melt additive can impart antistatic, hydrophilicity, and antimicrobial activity against bacteria to the dental appliance.

In some embodiments, the antimicrobial lipid compounds can be incorporated into a single layer dental appliance, or the antimicrobial lipid compounds can be incorporated into a selected layer of a multilayered dental appliance. In various embodiments, the dental appliance with the antimicrobial lipid compounds incorporated therein can have clarity, antimicrobial properties for odor reduction, resistance to hydration to enhance force persistence, and reduced coefficient of friction (static and/or dynamic) for patient comfort. In some embodiments, the antimicrobial properties of the dental appliance can persist for an extended period of time, even over the entire treatment time for which the dental appliance is to be used.

In some embodiments, the dental appliance is an orthodontic appliance, such as clear tray aligner or retainer, configured for moving or retaining the position of teeth in an upper or lower jaw of a patient.

The present disclosure is further generally directed to methods for incorporating an antimicrobial lipid compound or mixture of compounds in a layer or layers of an orthodontic appliance. For example, in one embodiment, an extruder may be used to form a polymeric sheet of material including the antimicrobial lipid compound or compounds, and the sheet can subsequently be thermoformed to have an arrangement of one or more cavities configured to receive one or more teeth.

In another aspect, the present disclosure is directed to a dental appliance including a polymeric shell with an arrangement of one or more cavities configured to receive one or more teeth, wherein the polymeric shell includes at least one antimicrobial lipid compound such as monolaurin. In some embodiments, the polymeric shell includes an outer layer with a first major external surface, an inner layer with a second major internal surface configured to contact the one or more teeth, and a core layer between the outer layer and the inner layer, and the antimicrobial lipid compounds may be incorporated into either or both of the outer layer and the inner layer.

In another aspect, the present disclosure is directed to a method including melt blending an antimicrobial lipid compound such as monolaurin with a first extrudable material to form a stream, extruding the stream to form a sheet, and shaping the sheet to form an arrangement of one or more cavities configured to receive one or more teeth.

In another aspect, the present disclosure is directed to a method including forming a polymeric shell including a plurality of cavities in a first major surface thereof, wherein the polymeric shell includes an antimicrobial lipid compound such as monolaurin, and wherein the cavities are configured to receive one or more teeth.

In another aspect, the present disclosure is directed to a method of orthodontic treatment, including positioning a dental appliance around one or more teeth, wherein the dental appliance includes a polymeric shell with an arrangement of one or more cavities configured to receive one or more teeth, wherein the polymeric shell includes an antimicrobial lipid compound or mixture of compounds, including compounds such as monolaurin.

The details of one or more embodiments of the techniques of this disclosure are set forth in the accompanying drawings and the description below. Other features, objects, and advantages of the techniques of this disclosure will be apparent from the description and drawings, and from the claims.

### BRIEF DESCRIPTION OF DRAWINGS

FIG. 1 is a schematic overhead perspective view of a dental alignment tray.

FIG. 2 is a schematic overhead perspective view of a method for using a dental alignment tray by placing the dental alignment tray to overlie teeth.

FIGS. 3A-3D are schematic cross-sectional views of an example orthodontic appliance.

Like symbols in the drawings indicate like elements.

### DETAILED DESCRIPTION

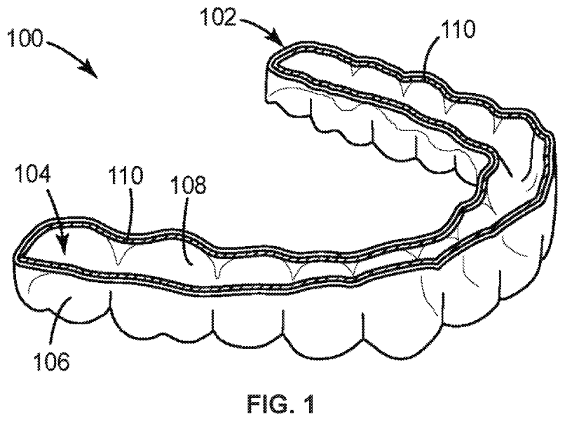

In one aspect, an orthodontic appliance 100 as shown in FIG. 1, which is also referred to herein as an orthodontic aligner tray, includes a thin polymeric shell 102 having a plurality of cavities 104 shaped to receive and resiliently reposition one or more teeth from one tooth arrangement to a successive tooth arrangement. Or, in the case of a retainer tray the thin polymeric shell 102 having a plurality of cavities 104 are shaped to receive and maintain the position of the previously realigned one or more teeth. The shell 102 includes cavities 104 configured to fit over one or more of the teeth present in the upper or lower jaw of a patient. A layer construction 110 includes a first major external surface 106 of the shell 102 and a second major internal surface 108 of the shell 102 that contacts the teeth of the patient. In some embodiments, the layer construction 110 of the shell 102 can be made of a single polymeric layer as well as multiple layers of different polymeric materials.

The shell 102 of the orthodontic appliance 100 is an elastic polymeric material that generally conforms to a patient's teeth, and may be transparent, translucent, or opaque. In some embodiments, the shell 102 is a clear or substantially transparent polymeric material that may include, for example, one or more of amorphous thermoplastic polymers, semi-crystalline thermoplastic polymers and transparent thermoplastic polymers chosen from polycarbonate, thermoplastic polyurethane, acrylic, polysulfone, polyprolylene, polypropylene/ethylene copolymer, cyclic olefin polymer/copolymer, poly-4-methyl-1-pentene or polyester/polycarbonate copolymer, styrenic polymeric materials, polyamide, polymethylpentene, polyetheretherketone and combinations thereof. In another embodiment, the shell 102 may be chosen from clear or substantially transparent semi-crystalline thermoplastic, crystalline thermoplastics and composites, such as polyamide, polyethylene terephthalate, polybutylene terephthalate, polyester/polycarbonate copolymer, polyolefin, cyclic olefin polymer, styrenic copolymer, polyetherimide, polyetheretherketone, polyethersulfone, polytrimethylene terephthalate, parylene, and mixtures and combinations thereof. In some embodiments, the shell 102 is a polymeric material chosen from polyethylene terephthalate, polyethylene terephthalate glycol (PETg), polycyclohexylenedimethylene terephthalate glycol (PCTg), and mixtures and combinations thereof.

Examples of commercially available materials suitable as the elastic polymeric material for the shell 102, which is not intended to be limiting, are PETg, PCTg, copolyester, polyolefin, thermoplastic polyurethane (TPU) and ethylene copolymer. Suitable polymeric materials can be obtained from various commercial suppliers such as, for example, Eastman Chemical, Kingsport, Tenn.; SK Chemicals, Irvine, Calif.; DowDuPont, Midland, Mich.; Pacur, Oshkosh, Wis.; Scheu Dental Tech, Iserlohn, Germany; Mitsui Chemicals, Tokyo, Japan. For example, materials available under the trade designation Tritan (available from Eastman Chemical) can be used to supply copolyester; materials available under the trade designation Neostar Elastomer FN007 (available from Eastman Chemical) can be used to supply copolyester ethers; materials available under the trade designation Elvaloy (available from DowDuPont, Midland, Mich.) can be used to supply copolymer resins, including ethylene copolymer; materials available under the trade designation Admer adhesive resins (available from Mitsui Chemicals, Tokyo, Japan) can be used to supply modified polyolefin with functional groups; and materials available under the trade designation Versaflex (available from PolyOne, Avon Lake, Ohio) can be used to supply thermoplastic elastomers (TPE).

In one embodiment, the shell 102 is a substantially transparent polymeric material. In this application, the term substantially transparent refers to materials that pass light in the wavelength region sensitive to the human eye (about 400 nanometer (nm) to about 750 nm) while rejecting light in other regions of the electromagnetic spectrum. In some embodiments, the reflective edge of the polymeric material selected for the shell 102 should be above about 750 nm, just out of the sensitivity of the human eye. In various embodiments, the visible light transmission through the thickness of the shell 102 is at least about 50%, or about 75%, or about 85%, or about 90%, or about 95%.

In the present application, the term “effective amount” means the amount of the antimicrobial lipid compound or compounds (e.g., monolaurin) when in a composition, as a whole, provides an antimicrobial (including, for example, antibacterial, antibiofilm, anti-plaque or antiseptic) activity that reduces, prevents, or eliminates one or more species of microbes (e.g., reduction of microbes to an acceptable level or an acceptable level of reduction). In some embodiments, the antimicrobial lipid compound can reduce microbes to a sufficiently low level to reduce odors from the antimicrobial device, including a non-detectable microbe level. In some embodiments, the antimicrobial lipid compound can provide an acceptable level of microbe reduction for the dental appliance, e.g., a 1-log reduction of S. mutans after 24-hour contact or a 3-log reduction of S. mutans after 24-hour contact.

The dental appliance described in the present disclosure can include a wide variety of antimicrobial lipid compounds to provide the antimicrobial effect. In one particularly preferred embodiment, the antimicrobial lipid compound or mixture of compounds includes an antimicrobial component.

The antimicrobial component content in the techniques of this disclosure (as it is ready to use) is typically at least 1 weight percent (wt %), 2 wt %, 5 wt %, 10 wt % and sometimes greater than 15 wt % of the total weight of the polymeric shell. The antimicrobial component may include one or more fatty acid esters of a polyhydric alcohol, fatty ethers of a polyhydric alcohol, or alkoxylated derivatives thereof (of either or both of the ester and/or ether), or combinations thereof. The term “fatty” means a straight or branched chain alkyl or alkylene moiety having 6 to 22 (odd or even number) carbon atoms, unless otherwise specified.

More specifically, the antimicrobial component is selected from the group of antimicrobial lipids consisting of a (C7-C12) saturated fatty acid ester of a polyhydric alcohol (preferably, a (C8-C12) saturated fatty acid ester of a polyhydric alcohol), an (C8-C22) unsaturated fatty acid ester of a polyhydric alcohol (preferably, an (C12-C22) unsaturated fatty acid ester of a polyhydric alcohol), a (C7-C12) saturated fatty ether of a polyhydric alcohol (preferably, a (C8-C12) saturated fatty ether of a polyhydric alcohol), an (C8-C22) unsaturated fatty ether of a polyhydric alcohol (preferably, an (C12-C22) unsaturated fatty ether of a polyhydric alcohol), an alkoxylated derivative thereof, and combinations thereof. Preferably, the esters and ethers are monoesters and monoethers, unless they are esters and ethers of sucrose in which case they can be monoesters, diesters, monoethers, or diethers. Various combinations of monoesters, diesters, monoethers, and diethers can be used in a composition of the techniques of this disclosure.

Preferably, the (C7-C12) saturated and (C8-C22) unsaturated monoesters and monoethers of polyhydric alcohols can be at least 80% pure (having 20% or less diester and/or triester or diether and/or triether), more preferably 85% pure, even more preferably 90% pure, most preferably 95% pure. Impure esters or ethers would not have sufficient, if any, antimicrobial activity.

Useful fatty acid esters of a polyhydric alcohol may have the formula:

(R¹—C(O)—O)ₙ—R²,

wherein R¹ is the residue of a (C7-C12) saturated fatty acid (preferably, a (C8-C12) saturated fatty acid), or a (C8-C22) unsaturated (preferably, a C12-C22) unsaturated, including polyunsaturated) fatty acid, R² is the residue of a polyhydric alcohol (typically and preferably, glycerin, propylene glycol, and sucrose, although a wide variety of others can be used including pentaerythritol, sorbitol, mannitol, xylitol, etc.), and n=1 or 2. The R² group includes at least one free hydroxy 1 group (preferably, residues of glycerin, propylene glycol, or sucrose). Preferred fatty acid esters of polyhydric alcohols are esters derived from C7, C8, C9, C10, C11, and C12 saturated fatty acids. For embodiments in which the polyhydric alcohol is glycerin or propylene glycol, n=1, although when it is sucrose, n=1 or 2. In general, monoglycerides derived from C10 to C12 fatty acids are food grade materials and GRAS materials.

Fatty acid esters are particularly useful candidates for treating surfaces exposed to microbes and pathogens such as those present in the oral environment. Many of the monoesters have been reported to be food grade, generally recognized as safe (GRAS) materials. For example, Kabara, J. of Food Protection. 44:633-647 (1981) and Kabara, J. of Food Safety. 4:13-25 (1982) report that LAURICIDIN (the glycerol monoester of lauric acid commonly referred to as monolaurin), a food grade phenolic and a chelating agent may be useful in designing preservative systems.

Fatty acid monoesters, such as glycerol monoesters of lauric, caprylic, capric, and heptanoic acid and/or propylene glycol monoesters of lauric, caprylic, capric and heptanoic acid, are active against Gram positive bacteria, fungi, yeasts and lipid coated viruses but alone are not generally active against Gram negative bacteria. When the fatty acid monoesters are combined with the enhancers described below, the composition is active against Gram negative bacteria.

Exemplary fatty acid monoesters include, but are not limited to, glycerol monoesters of lauric (monolaurin), caprylic (monocaprylin), and capric (monocaprin) acid, and propylene glycol monoesters of lauric, caprylic, and capric acid, as well as lauric, caprylic, and capric acid monoesters of sucrose. Other fatty acid monoesters include glycerin and propylene glycol monoesters of oleic (18:1), linoleic (18:2), linolenic (18:3), and arachonic (20:4) unsaturated (including polyunsaturated) fatty acids. As is generally known, 18:1, for example, means the compound has 18 carbon atoms and 1 carbon-carbon double bond. Preferred unsaturated chains have at least one unsaturated group in the cis isomer form. In certain preferred embodiments, the fatty acid monoesters that are suitable for use in the present composition include known monoesters of lauric, caprylic, and capric acid, such as that known as GML or the trade designation LAURICIDIN (the glycerol monoester of lauric acid commonly referred to as monolaurin or glycerol monolaurate), glycerol monocaprate, glycerol monocaprylate, propylene glycol monolaurate, propylene glycol monocaprate, propylene glycol monocaprylate, and combinations thereof.

Exemplary fatty acid diesters of sucrose include, but are not limited to, lauric, caprylic, and capric diesters of sucrose as well as combinations thereof.

A fatty ether of a polyhydric alcohol may have the formula:

(R³—O)ₙ—R⁴,

wherein R³ is a (C7-C12) saturated aliphatic group (preferably, a (C8-C12) saturated aliphatic group), or a (C8-C22) unsaturated (preferably, (C12-C22) unsaturated, including polyunsaturated) aliphatic group, R⁴ is the residue of a polyhydric alcohol. Preferred polyhydric alcohols include glycerin, sucrose, or propylene glycol. For glycerin and propylene glycol n=1, and for sucrose n=1 or 2. Preferred fatty ethers are monoethers of (C7-C12) alkyl groups (more preferably, (C8-C12) alkyl groups).

Exemplary fatty monoethers include, but are not limited to, laurylglyceryl ether, caprylglycerylether, caprylylglyceryl ether, laurylpropylene glycol ether, caprylpropyleneglycol ether, and caprylylpropyleneglycol ether. Other fatty monoethers include glycerin and propylene glycol monoethers of oleyl (18:1), linoleyl (18:2), linolenyl (18:3), and arachonyl (20:4) unsaturated and polyunsaturated fatty alcohols. In certain preferred embodiments, the fatty monoethers that are suitable for use in the present composition include laurylglyceryl ether, caprylglycerylether, caprylyl glyceryl ether, laurylpropylene glycol ether, caprylpropyleneglycol ether, caprylylpropyleneglycol ether, and combinations thereof. Unsaturated chains preferably have at least one unsaturated bond in the cis isomer form.

The alkoxylated derivatives of the aforementioned fatty acid esters and fatty ethers (e.g., one which is ethoxylated and/or propoxylated on the remaining alcohol groups) also have antimicrobial activity as long as the total alkoxylate is kept relatively low. Preferred alkoxylation levels are disclosed in U.S. Pat. No. 5,208,257. If the esters and ethers are ethoxylated, total moles of ethylene oxide are preferably less than 5, more preferably less than 2.

The fatty acid esters or fatty ethers of polyhydric alcohols can be alkoxylated, preferably ethoxylated and/or propoxylated, by conventional techniques. Alkoxylating compounds are preferably selected from the group consisting of ethylene oxide, propylene oxide, and mixtures thereof, and similar oxirane compounds.

The techniques of this disclosure typically include, a total amount of antimicrobial lipids such as fatty acid esters, fatty ethers, alkoxylated fatty acid esters, or alkoxylated fatty ethers of at least 1 wt %, at least 2 wt %, greater than 5 wt %, at least 6 wt %, at least 7 wt %, at least 10 wt %, at least 15 wt %, or at least 20 wt %, based on the total weight of the ready-to-use device. The term “ready-to-use” means the device in its intended form for use.

Antimicrobial lipids used in the techniques of this disclosure that include one or more fatty acid monoesters, fatty monoethers, hydroxyl acid esters of alcohols or alkoxylated derivatives thereof can also include a small amount of a di- or ti-fatty acid ester (i.e., a fatty acid di- or tri-ester), a di- or tri-fatty ether (i.e., a fatty di- or ti-ether), or alkoxylated derivative thereof. Preferably, such components comprise no more than 10 wt %, no more than 7 wt %, no more than 6 wt %, or no more than 5 wt %, of the total weight of the antimicrobial component. Thus, the monoester purity of the fatty acid monoester, fatty monoethers, hydroxyl acid esters of alcohols or alkoxylated derivatives thereof should exceed 85%, preferably 90%, and more preferably 95%. For example, for monoesters, monoethers, or alkoxylated derivatives of glycerin, preferably there is no more than 10 wt %, no more than 7 wt %, no more than 6 wt %, or no more than 5 wt % of a diester, diether, triester, triether, or alkoxylated derivatives thereof present, based on the total weight of the antimicrobial (e.g., monoester or monoether) components present in the composition. Preferably, the triester or diester content is kept low to preserve the antimicrobial efficacy of the antimicrobial component.

An additional class of antimicrobial lipid is a fatty alcohol ester of a hydroxyl functional carboxylic acid preferably of the formula:

R⁵—O—(—C(O)—R⁶—O)ₙH,

wherein R⁵ is the residue of a (C7-C14) saturated alkyl alcohol (preferably, a (C7-C12) saturated alkyl alcohol, more preferably, a (C8-C12) saturated alkyl alcohol) or a (C8-C22) unsaturated alcohol (including polyunsaturated alcohol), R⁶ is the residue of a hydroxycarboxylic acid wherein the hydroxycarboxylic acid has the following formula:

R⁷(CR⁸OH)ₚ(CH₂)_{q}COOH,

wherein: R⁷ and R⁸ are each independently H or a (C1-C8) saturated straight, branched, or cyclic alkyl group, a (C6-C12) aryl group, or a (C6-C12) aralkyl or alkaryl group wherein the alkyl groups, are saturated straight, branched, or cyclic, wherein R⁷ and R⁸ may be optionally substituted with one or more carboxylic acid groups; p=1 or 2; and q=0-3; and n=1, 2, or 3. The R6 group may include one or more free hydroxyl groups but preferably is free of hydroxyl groups. Preferred fatty alcohol esters of hydroxycarboxylic acids are esters derived from branched or straight chain C8, C9, C10, C11, or C12 alkyl alcohols. The hydroxyacids typically have one hydroxyl group and one carboxylic acid group.

In one aspect, the antimicrobial lipid component includes a (C7-C14) saturated fatty alcohol monoester of a (C2-C8) hydroxycarboxylic acid (preferably, a (C7-C12) saturated fatty alcohol monoester of a (C2-C8) hydroxycarboxylic acid, more preferably a (C8-C12) saturated fatty alcohol monoester of a (C2-C8) hydroxycarboxylic acid), a (C8-C22) mono- or poly-unsaturated fatty alcohol monoester of a (C2-C8) hydroxycarboxylic acid, an alkoxylated derivative of either of the foregoing, or combinations thereof. The hydroxycarboxylic acid moiety can include aliphatic and/or aromatic groups. For example, fatty alcohol esters of salicylic acid are possible. As used herein, a “fatty alcohol” is an alkyl or alkylene monofunctional alcohol having an even of odd number of carbon atoms.

Exemplary fatty alcohol monoesters of hydroxycarboxylic acids include, but are not limited to, (C6-C12) fatty alcohol esters of lactic acid such as octyl lactate, 2-ethylhexyl lactate (Purasolv EIIL from Purac, Lincolnshire Ill., lauryl lactate (Chrystaphyl 98 from Chemic Laboratories, Canton Mass.), lauryl lactyl lacate, 2-ethylhexyl lactyl lactate; (C8-C12) fatty alcohol esters of glycolic acid, lactic acid, 3-hydroxybutanoic acid, mandelic acid, gluconic acid, tartaric acid, and salicylic acid.

The alkoxylated derivatives of the fatty alcohol esters of hydroxy functional carboxylic acids (e.g., one which is ethoxylated and/or propoxylated on the remaining alcohol groups) also have antimicrobial activity as long as the total alkoxylate is kept relatively low. The preferred alkoxylation level is less than 5 moles, and more preferably less than 2 moles, per mole of hydroxycarboxylic acid.

The above antimicrobial lipid components comprising an ester linkage are hydrolytically sensitive, and may be degraded by exposure to water, particularly at extreme pH (less than 4 or more than 10) or by certain bacteria that can enzymatically hydrolyze the ester to the corresponding acid and alcohol, which may be desirable in certain applications. For example, an article may be made to degrade rapidly by incorporating an antimicrobial lipid component comprising at least one ester group. If extended persistence of the article is desired, an antimicrobial lipid component, free of hydrolytically sensitive groups, may be used. For example, the fatty monoethers are not hydrolytically sensitive under ordinary processing conditions and are resistant to microbial attack.

Another class of antimicrobial components includes cationic amine antimicrobial compounds, which include antimicrobial protonated tertiary amines and small molecule quaternary ammonium compounds. Exemplary small molecule quaternary ammonium compounds include benzalkonium chloride and alkyl substituted derivatives thereof, di-long chain alkyl (C8-C18) quaternary ammonium compounds, cetylpyridinium halides and their derivatives, benzethonium chloride and its alkyl substituted derivatives, octenidine and compatible combinations thereof.

Cationic antiseptics and disinfectants useful as the antimicrobial component include small molecule quarternary ammonium compounds, typically comprising one or more quaternary ammonium group, having attached thereto at least one C6-C18 linear or branched alkyl or aralkyl chain. Suitable compounds include those disclosed in Disinfection, Sterilization and Preservation, S. Block, 4th ed., 1991, Chapter 13, Lea & Febiger, and may have the formula:

R⁹R¹⁰NR¹¹R¹²⁺X⁻,

in which R⁹ and R¹⁰ are C1-C18 linear or branched alkyl, alkaryl, or aralkyl chains that may be substituted by N, O or S provided at least one R⁹ or R¹⁰ is a C8-C18 linear of branched alkyl, alkaryl, or aralkyl moiety that may be substituted by N, O or S, R¹¹ and R¹² are C1-C6 alkyl, phenyl, benzyl or C8-C12 alkaryl groups, or R¹¹ and R¹² may form a ring such as a pyridine ring with the N of the quaternary ammonium group, X is an anion, preferably halide such as Cl⁻ or Br⁻ but possibly methosulfate, ethosulfate, phosphate or similar anions. Compounds within this class are: monoalkyltrimethylammonium salts, monoalkyldimethyl-benzyl ammonium salts, dialkyldimethyl ammonium salts, behzethonium chloride, alkyl substituted benzethonium halides such as methylbenzethonium chloride and octenidine.

Examples of quaternary ammonium antimicrobial components are: benzalkonium halides having an alkyl chain length of C8-C18, preferably C12-C16, more preferably a mixture of chain lengths, e.g., benzalkoniumchloride having 40% C12 alkyl chains, 50% C14 alkyl chains, and 10% C16 chains (available under the trade designation Barquat MB-50 from Lonza Group Ltd., Basel, Switzerland); benzalkonium halides substituted with alkyl groups oh the phenyl ring (available under the trade designation as Barquat 4250 from Lonza); dimethyldialkylammonium halides having C8-C18 alkyl groups, or mixtures of such compounds (available under the trade designation as Bardac 2050, 205M and 2250 from Lonza); and cetylpyridinium halides such as cetylpyridinium chloride (available under the trade designation as Cepacol Chloride from Merrell Labs); benzethonium halides and alkyl substituted benzethonium halides (available under the trade designation as Hyamine 1622 and Hyamine 10× from Rohm and Haas).

A useful class of cationic antimicrobials is based on protonated tertiary amines. Preferred cationic antimicrobial protonated tertiary amines have at least one C6-C18 alkyl group. Within this class are biodegradable derivatives of amino acids, as described in PCT publications WO 01/94292, WO 03/013454 and WO 03/034842, and combinations of those with sodium sorbate, potassium sorbate of sorbic acid, see WO 02/087328. These cationic antimicrobial components can be degraded in the environment or on living tissue. WO 03/013454 teaches such antimicrobial components having the formula:

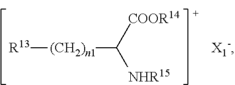

in which X may be Br⁻, Cl⁻ or HSO₄⁻, R¹⁵ may be a straight C8-C14 alkyl chain from an acid, e.g., saturated fatty hydroxy acid, R¹⁴ is a C1-C18 straight chain or branched alkyl or an aromatic moiety; and R¹³ may be —NH3,

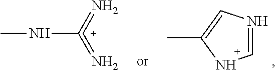

and n1 may be 0-4.

One useful member of this class of materials is lauroylethylarginate (the ethyl ester and lauric acid amide of the amino acid arginine (available under the trade designation as Mirenat N from A&B Ingredients, Fairfield, N.J.)). Methods for producing these compositions are disclosed in WO 01/94292.

The cationic antimicrobial components are typically added to the compositions of the techniques of this disclosure at a concentration of at least 1.0 wt %, preferably at least 3 wt %, more preferably greater than 5.0 wt %, still more preferably at least 6.0 wt %, even more preferably at least 10 wt % and most preferably at least 20.0 wt %, in some cases exceeding 25 wt %. Preferably, the concentration is less than 50 wt %, more preferably less than 40 wt %, and most preferably less than 35 wt %. Lower levels may be possible when used in combination with certain enhancers such as sorbic acid and/or its salts.

The antimicrobial components of the techniques of this disclosure may be used alone or in combination in order to effectively kill microorganisms. Combinations of antimicrobial components that result in unstable compositions or that arc incompatible with each other should be avoided. For example, quaternary ammonium compounds may be incompatible with alkyl carboxylic acids or surfactants containing a sulfate moiety and/or sulfonic acid, and certain salts may cause precipitation of quaternary ammonium compounds.

As used herein, the term “antiseptic” refers to a substance that inhibits growth and reproduction of disease-causing microorganisms, especially those substances that may contact mammalian tissue such as skin, wounds, mucosal tissue and the like. In most cases, “antiseptic” is synonymous with antimicrobial when used to control mammalian pathogens. Antiseptics and antimicrobial components described herein may be used alone, in combination, or with other antimicrobial components. Additional antimicrobial components for use with those already described include peroxides, C6-C14 alkyl carboxylic acids and alkyl ester carboxylic acids, antimicrobial natural oils, polymeric biguanides (such as polyhexamethylene biguanide) and bisbiguanides (such as chlorhexidine and its salts including chlorhexidine gluconate) and compatible combinations thereof as mentioned in U.S. Patent Publication 2006/0051384. Other compatible antiseptics that may be used in combination with the compositions of the techniques this disclosure on surfaces are iodine, iodophors, antimicrobial metals and metal salts such as silver salts and silver oxide, copper and zinc salts. In addition, certain antibiotics may be blended into the compositions of the techniques of this disclosure or coated on the surface of articles comprising them and include the antibiotic available under the trade designation as Neosporin (available from Johnson & Johnson, New Brunswick, N.J.), polymyxin, bacitracin, mupirocin, rifampin, minocycline, tetracycline, beta lactam antibiotics such as penicillin, methicillin and amoxicillin, fluoroquinolones, clindamycin, cephalosporins, macrolides, and aminoglycosides.

The term “enhancer” means a component that enhances the effectiveness of the antimicrobial component such that when the composition without the enhancer is used separately, it does not provide the same level of antimicrobial activity as the composition including enhancer. An enhancer in the absence of the antimicrobial component may not provide any appreciable antimicrobial activity. The enhancing effect can be with respect to the level of kill, the speed of kill, and/or the spectrum of microorganisms killed, and may not be seen for all microorganisms. In fact, an enhanced level of kill is most often seen in Gram negative bacteria such as Escherichia coli. An enhancer may be a synergist such that when combined with the remainder of the composition, the combined antimicrobial activity is greater than the sum of the activity of the composition without the enhancer component and the composition without the antimicrobial component.

The compositions of the techniques of this disclosure may include an enhancer (preferably a synergist) to enhance the antimicrobial activity especially against Gram negative bacteria, e.g., E. coli and Pseudomonas sp. The chosen enhancer preferably affects the cell envelope of the bacteria. While not wishing to be bound by theory, it is presently believed that the enhancer functions by allowing the antimicrobial component to more easily enter the cell cytoplasm and/or by facilitating disruption of the cell envelope. The enhancer component may include an alpha-hydroxy acid, a beta-hydroxy acid, other carboxylic acids, a (C2-C6) saturated or unsaturated alkyl carboxylic acid, a (C6-C16) aryl carboxylic acid, a (C6-C16) aralkyl carboxylic acid, a (C6-C12) alkaryl carboxylic acid, a phenolic compound (such as certain antioxidants and parabens), a (C5-C10) monohydroxy alcohol, a chelating agent, a glycol ether (i.e., ether glycol), or oligomers that degrade to release one of the above enhancers. Examples of such oligomers are oligomers of glycolic acid, lactic acid or both having at least 6 repeat units. Various combinations of enhancers can be used if desired.

The alpha-hydroxy acid, beta-hydroxy acid, and other carboxylic acid enhancers are preferably present in their protonated, free acid form. It is not necessary for all of the acidic enhancers to be present in the free acid form; however, the preferred concentrations listed below refer to the amount present in the free acid form. Additional, non-alpha hydroxy acid, betahydroxy acid or other carboxylic acid enhancers, may be added in order to acidify the formulation or buffer it at a pH to maintain antimicrobial activity. Preferably, acids are used having a pKa greater than about 2.5, preferably greater than about 3, and most preferably greater than about 3.5 in order to avoid hydrolyzing the aliphatic polyester component. Furthermore, chelator enhancers that include carboxylic acid-groups are preferably present with at least one, and more preferably at least two, carboxylic acid groups in their free acid form. The concentrations given below assume this to be the case. The enhancers in the protonated acid form are believed to not only increase the antimicrobial efficacy, but to improve compatibility when incorporated into the aliphatic polyester component.

One or more enhancers may be used in the compositions of the of the techniques of this disclosure at a suitable level to produce the desired result. Enhancers are typically present in a total amount greater than 0.1 wt %, preferably in an amount greater than 0.25 wt %, more preferably in an amount greater than 0.5 wt %, even more preferably in an amount greater than 1.0 wt %, and most preferably in an amount greater than 1.5 wt % based on the total weight of the ready-to-use composition. In a preferred embodiment, they are present in a total amount of no greater than 10 wt %, based on the total weight of the ready to use composition. Such concentrations typically apply to alpha-hydroxy acids, beta-hydroxy acids, other carboxylic acids, chelating agents, phenolics, ether glycols, and (C5-C10) monohydroxy alcohols.

The ratio of the enhancer component relative to the total concentration of the antimicrobial component is preferably within a range of 10:1 to 1:300, and more preferably 5:1 and 1:10, on a weight basis.

The alpha-hydroxy acid is typically a compound of the formula:

R¹⁶(CR¹⁷OH)ₙ₂COOH,

wherein: R¹⁶ and R¹⁷ are each independently H or a (C1-C8) alkyl group (straight, branched,

or cyclic), a (C6-C12) aryl, or a (C6-C12) aralkyl or alkaryl group (wherein the alkyl group is straight, branched, or cyclic), R¹⁶ and R¹⁷ may be optionally substituted with one or more carboxylic acid groups; and n2=1-3, preferably, n2=1-2.

Exemplary alpha-hydroxy acids include, but are not limited to, lactic acid, malic acid, citric acid, 2-hydroxybutanoic acid, 3-hydroxybutanoic acid, mandelic acid, gluconic acid, glycolic acid, tartaric add, alpha-hydroxyethanoic acid, ascorbic acid, alpha-hydroxyoctanoic acid, and hydroxycaprylic: acid, as well as derivatives thereof (e.g., compounds substituted with hydroxyls, phenyl groups, hydroxyphenyl groups, alkyl groups, halogens, as well as combinations thereof). Preferred alpha-hydroxy acids include lactic acid, malic acid, and mandelic acid. These acids may be in D, L, or DL form and may be present as free acid, lactone, or partial salts thereof. All such forms are encompassed by the term “acid.” Preferably, the acids are present in the free acid form. In certain preferred embodiments, the alpha-hydroxy acids useful in the compositions of the of the techniques of this disclosure, are selected from the group consisting of lactic acid, mandelic acid, malic acid, and mixtures thereof. Other suitable alpha-hydroxy acids are described in U.S. Pat. No. 5,665,776 (Yu).

One or more alpha-hydroxy acids may be incorporated in the compositions of the techniques of this disclosure, and/or applied to the surfaces of articles comprising the device of this disclosure, in an amount to produce the desired result. They may be present in a total amount of at least 0.25 wt %, at least 0.5 wt %, and at least 1 wt %, based on the total weight of the ready-to-use composition. They may be present in a total amount of no greater than 10 wt %, no greater than 5 wt %, or no greater than 3 wt %, based on the total weight of the ready-to-use composition.

The weight ratio of alpha-hydroxy acid enhancer to total antimicrobial lipid component is at most 50:1, at most 30:1, at most 20:1, at most 10:1, at most 5:1 or at most 1:1. The ratio of alpha-hydroxy acid enhancer to total antimicrobial component may be at least 1:120, at least 1:80, or at least 1:60. Preferably the ratio of alpha-hydroxy acid enhancer to total antimicrobial component is within a range of 1:60 to 2:1.

A beta-hydroxy acid enhancer is typically a compound represented by the formula:

R¹⁸(CR¹⁹OH)ₙ₃(CHR²⁰)ₘCOOH or

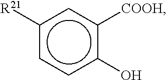

wherein: R¹⁸, R¹⁹, and R²⁰ are each independently H or a (C1-C8)alkyl group (saturated straight, branched, or cyclic group), (C6-C12) aryl, or (C6-C12) aralkyl or alkaryl group (wherein the alkyl group is straight, branched, or cyclic), R¹⁸ and R¹⁹ may be optionally substituted with one or more carboxylic acid groups; m=0 or 1; n3=1-3 (preferably, n3=1-2); and R²¹ is H, (C1-C4) alkyl or a halogen.

Exemplary beta-hydroxy acids include, but are/not limited to, salicylic acid, beta-hydroxybutanoic acid, tropic acid, and trethocanic acid. In certain preferred embodiments, the beta-hydroxy acids useful in the compositions of the techniques of this disclosure are selected from the group consisting of salicylic acid, beta-hydroxybutanoic acid, and mixtures thereof. Other suitable beta-hydroxy acids are described in U.S. Pat. No. 5,665,776.

One or more beta-hydroxy acids may be used in the compositions of the device of the techniques of this disclosure at a suitable level to produce the desired result. They may be present in a total amount of at least 0.1 wt %, at least 0.25 wt %, or at least 0.5 wt %, based on the total weight of the ready-to-use composition. They may also be present in a total amount of no greater than 10 wt %, no greater than 5 wt %, and no greater than 3 wt %, based on the total weight of the ready-to-use composition. Higher concentrations may become irritating to tissue. Alternatively, beta-hydroxy acids may be applied to the surface of articles comprising the composition of the techniques of this disclosure. When present on the surface, the levels may be 0.05 wt %, preferably 0.1 wt %, more preferably 0.25 wt %, and most, preferably 0.5 wt % of the dental appliance.

The weight ratio of beta-hydroxy acid enhancer to total antimicrobial lipid component is preferably at most 50:1, at most 30:1, at most 20:1, at most 10:1, at most 5:1, or at most 1:1. The ratio of beta-hydroxy acid enhancer to total antimicrobial lipid component is preferably at least 1:120, at least 1:80, or at least 1:60. Preferably the ratio of beta-hydroxy acid enhancer to total antimicrobial component is within a range of 1:60 to 2:1, more preferably 1:15 to 1:1.

In systems with low concentrations of water, or that are essentially free of water, transesterification may be the principle route of loss of the fatty acid monoester and alkoxylated derivatives of these active ingredients and loss of carboxylic acid containing enhancers may occur due to esterification. Thus, certain alpha-hydroxy acids (AHA) and beta-hydroxy acids (BHA) are particularly preferred since these are believed to be less likely to transesterify the ester antimicrobial or other ester by reaction of the hydroxyl group of the AHA or BHA. For example, salicylic acid may be particularly preferred in certain formulations since the phenolic hydroxyl group is a much more acidic alcohol and thus much less likely to react. Other particularly preferred compounds in anhydrous or low-water content formulations include lactic, mandelic, malic, citric, tartaric, and glycolic acid. Benzoic acid and substituted benzoic acids that do not include a hydroxyl group, while not hydroxyl acids, are also preferred due to a reduced tendency to form ester groups. This applies to both melt and solvent cast processable systems or compositions.

Carboxylic acids other than alpha- and beta-carboxylic acids are suitable enhancers. They include alkyl, aryl, aralkyl, or alkaryl carboxylic acids typically having equal to or less than 12 carbon atoms. A preferred class of these can be represented by the following formula:

R²²(CR²³₂)ₙ₂COOH,

wherein: R²² and R²³ are each independently H or a (C1-C4) alkyl group (which can be a straight, branched, or cyclic group), a (C6-C12) aryl group, a (C6-C12) group containing both aryl groups and alkyl groups (which can be a straight, branched, or cyclic group), R²² and R²³ may be optionally substituted with one or more carboxylic acid groups and n2=0-3, preferably, n2=0-2. The carboxylic acid may be a (C2-C6) alkyl carboxylic acid, a (C6-C16) aralkyl carboxylic acid, or a (C6-C16) alkaryl carboxylic acid. Exemplary acids include, but are not limited to acetic acid, propionic acid, sorbic acid, benzoic acid, benzylic acid, and nonylbenzoic acid.

One or more such carboxylic acids may be used in the compositions of the present application in amounts sufficient to produce the desired result. In certain embodiments, they are present in a total amount no greater than 5 wt %, preferably no greater than 3 wt %, based on the total weight of the ready-to-use composition.

Alternatively, carboxylic acid enhancers may be present on the surface of an article made from the composition of the techniques of this disclosure. When present on the surface, the amounts used may be 0.05 wt %, preferably 0.1 wt %, more preferably 0.25 wt %, and most preferably 0.5 wt % of the article.

The weight ratio of the total concentration of carboxylic acids (other than alpha- or beta-hydroxy acids) to the total concentration of the antimicrobial component is preferably within a range of 10:1 to 1:100, and preferably 2:1 to 1:10.

A chelating agent (i.e., chelator) is typically an organic compound capable of multiple coordination sites with a metal ion in solution. Typically, these chelating agents are polyanionic compounds and coordinate best with polyvalent metal ions. Exemplary chelating agents include, but are not limited to, ethylene diamine tetraacetic acid (EDTA) and salts thereof (e.g., EDTA(Na)₂, EDTA(Na)₄, EDTA(Ca), EDTA(K)₂), sodium acid pyrophosphate, acidic sodium hexametaphosphate, adipic acid, succinic acid, polyphosphoric acid, sodium acid pyrophosphate, sodium hexametaphosphate, acidified sodium hexametaphosphate, nitrilotris(methylenephosphonic acid), diethylenetriaminepentaacetic acid, 1-hydroxyethylene, 1,1-diphosphonic acid, and diethylenetriaminepenta-(methylenephosphonic acid). Certain carboxylic acids, particularly the alpha-hydroxy acids and beta-hydroxy acids, can also function as chelators, e.g., malic acid and tartaric acid.

Also included as chelators are compounds highly specific for binding ferrous and/or ferric ion such as siderophores, and iron binding proteins. Iron binding protein include, for example, lactoferrin, and transferrin. Siderophpres include, for example, enterochlin, enterobactin, vibriobactin, anguibactin, pyochelin, pyoverdin, and aerobaetin.

In certain embodiments, the chelating agents useful in the compositions of the techniques of this disclosure include those selected from the group consisting of ethylenediaminetetraacetic acid and salts thereof, succinic acid, and mixtures thereof. Preferably, either the free acid or the mono- or di-salt form of EDTA is used.

One or more chelating agents may be used in the compositions of the techniques of this disclosure at a suitable level to produce the desired result. They may be used in amounts similar to the carboxylic acids described above.

The ratio of the total concentration of chelating agents (other than alpha- or beta-hydroxy acids) to the total concentration of the antimicrobial component is preferably within a range of 1.0:1 to 1:100, and more preferably 1:1 to 1:10, on a weight basis.

A phenolic compound enhancer is typically a compound having the following general structure:

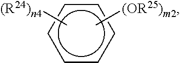

wherein: m2 is 0 to 3 (especially 1 to 3), n4 is 1 to 3 (especially 1 to 2), each R24 independently is alkyl or alkenyl of up to 12 carbon atoms (especially up to 8 carbon atoms) optionally substituted with O in of on the chain (e.g., as a carbonyl group) or OH on the chain, and each R²⁵ independently is H or alkyl or alkenyl of up to 8 carbon atoms (especially up to 6 carbon atoms) optionally substituted with O in or on the chain (e.g., as a carbonyl group) or OH on the chain, but if R²⁵ is H, n4 preferably is 1 or 2.

Examples of phenolic enhancers include, but are not limited to, butylated hydroxy anisole, e.g., 3(2)-tert-butyl-4-methoxyphenol (BHA), 2,6-di-tert-butyl-4-methylphenol (BHT), 3,5-di-tert-butyl-4-hydroxybenzylphenol, 2,6-di-tert-4-hexylphenol, 2,6-di-tert-4-octylphenol, 2,6-di-tert-4-decylphenol, 2,6-di-tert-butyl-4-ethylphenol, 2,6-di-tert-4-butylphenol, 2,5-di-tert-butylphenol, 3,5-di-tert-butylphenol, 4,6-di-tert-butyl-resorcinol, methyl paraben (4-hydroxybenzoic acid methyl ester), ethyl paraben, propyl paraben, butyl paraben, 2-phenoxyethanol, as well as combinations thereof. One group of the phenolic compounds is the phenol species having the general structure shown above where R²⁵ is H and where R²⁴ is alkyl or alkenyl of up to 8 carbon atoms, and n4 is 0, 1, 2, or 3, especially where at least one R²⁴ is butyl and particularly tert-butyl, and especially the non-toxic members thereof being preferred. Some of the phenolic synergists are BHA, BHT, methyl paraben, ethyl paraben, propyl paraben, and butyl paraben as well as combinations of these. One or more phenolic compounds may be used in the compositions of the techniques of this disclosure at a suitable level to produce the desired result. The concentrations of the phenolic compounds may vary widely, but typically greater than 0.5 wt %, based on the total weight of the composition, can be effective when the above-described esters are present within the above-noted ranges. In some embodiments, they are present in a total amount of at least 0.75 wt %, or at least 1.0 wt %, based on the total weight of the composition. In other embodiments, they are present in a total amount of no greater than 8 wt %, no greater than 4 wt %, or no greater than 2 wt %, based on the ready to use composition.

The weight ratio of the total phenolic concentration to the total concentration of the antimicrobial component may be within a range of 1:1 to 1:100, or preferably within a range of 1:1 to 1:10, on a weight basis.

The above-noted concentrations of the phenolics are normally observed unless concentrated formulations for subsequent dilution are intended. The minimum concentration of the phenolics and the antimicrobial components to provide an antimicrobial effect will vary with the particular application.

An additional enhancer is a monohydroxy alcohol having 5-10 carbon atoms, including C5-C10 monohydroxy alcohols (e.g., octanol and decanol). In certain embodiments, alcohols useful in the compositions of the techniques of this disclosure are selected from the group n-pentanol, 2 pentanol, n-hexanol, 2 methylpentyl alcohol, n-octanol, 2-ethylhexyl alcohol, decanol, and mixtures thereof.

C5-C10 alcohols may be present in a total amount of at least 1 wt %, at least 2 wt %, at least 3 wt %, or at least 5 wt %, based on the composition. C5-C10 alcohols may be present in a total amount of no greater than 20 wt %, no greater than 15 wt %, or no greater than 10 wt %, based on the total weight of the composition. C5-C10 alcohols may be applied to the surface of articles comprising the composition of polymer and antimicrobial component. When present on the surface, amounts may be at least 0.05 wt %, preferably at least 0.1 wt %, more preferably at least 0.25 wt %, and most preferably at least 0.5 wt % of the article to which the composition is applied.

An additional enhancer is an ether glycol. Exemplary ether glycols include those of the formula:

R′″—O—(CH₂CHR″″O)ₙ₅(CH₂CHR″″O)H,

wherein R′″=H, a (C1-C8) alkyl, or a (C6-C12) aralkyl or alkaryl; and each R″″ is independently=H, methyl, or ethyl; and n5=0-5, preferably 1-3. Examples include 2-phenoxyethanol, dipropylene glycol, triethylene glycol, the line of products available under the trade designation DOWANOL DB (di(ethylene glycol) butyl ether), DOWANOL DPM (di(propylene glycol)monomethyl ether), and DOWANOL TPnB (tri(propylene glycol) monobutyl ether), as well as many others available from Dow Chemical Company, Midland Mich.

One or more ether glycols may be present in a total amount of at least 0.5 wt %, based on the total ready-to-use composition. In an embodiment, they are present in a total amount of no greater than 20 wt %, based on the total weight of the ready-to-use composition. The ether glycols may be present on the surface of articles comprising the composition of the techniques of this disclosure. When present on the surface, the amounts may be at least 0.05 wt %, preferably at least 0.1 wt %, more preferably at least 0.25 wt %, and, most preferably at least 0.5 wt % of the articles to which the glycols are applied as part of the composition of the techniques of this disclosure.

Oligomers that release an enhancer may be prepared by a number of methods. For example, oligomers may be prepared from alpha hydroxy acids, beta hydroxy acids, or mixtures thereof by standard esterification techniques. Typically, these oligomers have at least two hydroxy acid units, preferably at least 10 hydroxy acid units, and most preferably at least 50 hydroxy acid units. For example, a copolymer of lactic acid and glycolic acid may be prepared as shown in the Examples section.

Alternatively, oligomers of (C2-C6) dicarboxylic acids and diols may be prepared by standard esterification techniques, these oligomers preferably have at least 2 dicarboxylic acid units; preferably at least 10 dicarboxylic acid units, and most preferably at least 50 dicarboxylic acid units.

The enhancer releasing oligomeric polyesters used typically have a weight average molecular weight of less than 10,000 daltons and preferably less than 8,000 daltons.

These oligomeric polyesters may be hydrolyzed. Hydrolysis can be accelerated by an acidic or basic environment, for example at a pH less than 5 or greater than 8. The oligomers may be degraded, enzymatically by enzymes present in the composition or in the environment in which it is used, for example from mammalian tissue or from microorganisms in the environment.

The compositions of the techniques of this disclosure may further include other additives, including surfactants and flavorants and flavor masking agents. For those compositions that include a major amount of the antimicrobial lipid that is liquid at room temperature, the antimicrobial lipid may serve as both the active antimicrobial agent and a vehicle for the other components of the antimicrobial composition.

The dental appliances described in the present disclosure can include a wide variety of antimicrobial lipid compounds to provide the antimicrobial effect. In one particularly preferred embodiment, the antimicrobial lipid compound or mixture of compounds includes the antimicrobial lipid known as monolaurin, which is a U.S. Food and Drug Administration (FDA) approved food additive and an antimicrobial compound with biological activity. Monolaurin has been used in various food products and has also been used as a dietary supplement with positive effect on the human immune system. Monolaurin has also been used as an ingredient in disinfection and coating or topical treatment for healthcare.

Monolaurin has the general structure of Formula I:

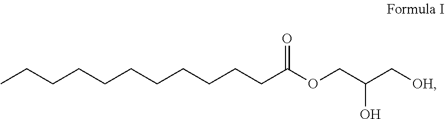 [SMILES: CCCCCCCCCCCC(=O)OCC(O)CO]

Suitable monolaurin compounds include, but are not limited to, 1-Mono-laurin; (±)-2,3-Dihydroxypropyl dodecanoate; (±)-Glyceryl 1-monododecanoate; 1-Glyceryl laurate; 1-Monododecanoylglycerol; 1-Monolaurin; 1-Monolauroyl-rac-glycerol; 1-lauroyl-glycerol; Monolauroylglycerin; 3-Dodecanoyloxy-1,2-propanediol; Dodecanoic acid α-monoglyceride; Glycerin 1-monolaurate; Glycerol 1-laurate; Glycerol 1-monododecanoate; Glycerol 1-monolaurate; Glycerol α-monolaurate; Glyceryl monolaurate; Glyceryl laurate; Glyceryl monododecanoate; Lauric acid 1-monoglyceride; Lauric acid α-monoglyceride; Lauricidin; Luaricidin; NSC 698570; α-Monolaurin, and mixtures and combinations thereof.

In some embodiments, the monolaurin can be used with an additional antimicrobial compound to provide an enhanced antimicrobial effect. In various embodiments, which are not intended to be limiting, additional antimicrobial compounds that may be utilized with monolaurin include metals, metal oxides, cationic surfactants free of aromatic groups, cationic antimicrobial polymers, antimicrobial lipids, and alkyl carboxylic acids and alkyl carboxylate ester carboxylic acids. In some embodiments, the additional antimicrobial compounds can be generated in-situ by exposure to ultraviolet (UV) light.

Metals and metal oxides have also been shown to have an antimicrobial effect. In one example, silver, particularly the ion Ag⁺, can be an effective antiseptic and has been used in creams to treat wounds and other topical infections. The silver ions can be delivered from a variety of salts and complexes including silver zeolites; inorganic silver salts (e.g., silver nitrate, silver chloride, silver sulfate, silver thiosulfate; silver phosphate, silver alkyl, silver aryl, and silver aralkyl carboxylates (exemplary carboxylate anions have less than about 8 carbon atoms (e.g., the acetate, lactate, salicylate, and gluconate salts)); silver oxide, colloidal silver, nanocrystalline silver, silver coated microspheres, silver complexed with various polymers as well as silver delivered from dendrimers as described, for example, in U.S. Pat. Nos. 6,579,906 and 6,224,898, the disclosures of which are incorporated herein by reference; and silver antimicrobial complexes (e.g., silver sulfadiazine). The silver can be complexed with primary, secondary, tertiary, and quaternary amines as well as polymeric forms thereof, and silver protein complexes.

Other antimicrobial metals include, but are not limited to, copper (Cu2+) and zinc (Zn2+), and the same types of salts and complexes referred to above for silver can also be used to deliver copper and zinc ions to provide an antimicrobial effect.

Those compounds of this class that may be, for example, vacuum deposited may be coated on the external surface of the device as described U.S. Pat. No. 9,393,350, the disclosure of which is incorporated herein by reference.

In other embodiments, antimicrobial polymers including quaternary amine or protonated primary, secondary, or tertiary amine groups can be used to provide antimicrobial or antiseptic properties. In one embodiment, to further provide UV stability, the cationic antimicrobial polymer can be a polyquaternary amine, which has quaternary amine groups with at least one alkyl or aralkyl chain of at least 6 carbon atoms, and preferably as least 8 carbon atoms. The polymers can be linear, branched, hyperbranched or dendrimers. Exemplary antimicrobial polymeric quaternary amine polymers include those described in U.S. Pat. Nos. 6,440,405, 5,408,022, and 5,084,096; PCT Pub. No. WO/02102244; and Disinfection, Sterilization and Preservation, S. Block, 4th ed., 1991, Chapter 13, Lea & Febiger, the disclosures of which are incorporated herein by reference.

In another embodiment, one class of polymeric protonated amine antiseptic compounds that can be utilized in the dental appliance are polybiguanides. Compounds of this class are represented by the formula:

X—R¹—NH—C(NH)—NH—C(NH)—NH—R²—NHC(NH)—NH—C(NH)—NH—R³—X,

where R¹, R², and R³ are bridging groups, such as polymethylene groups (in some embodiments, having 2 to 10, 4 to 8, or 6 methylene groups). The methylene groups can be substituted in available positions with halogen, hydroxyl, or phenyl groups. X is a terminal group and can be an amine, amine salt, or a dicyandiamide group. One compound of this class is polyhexamethylenebiguanide (PHMB) (available, for example, under the trade designation COSMOCIL CQ from Aveci, Wilmington, Del.).

In some embodiments, an antimicrobial cationic surfactant can also be used with the antimicrobial compounds. This class of compounds can include at least one quaternary ammonium group, wherein attached to the quaternary ammonium group is at least one C6-C18 linear or branched alkyl chain. Suitable compounds include, but are not limited to, those disclosed in Disinfection, Sterilization and Preservation, S. Block, 4th ed., 1991, Chapter 13, Lea & Febiger. Compounds of this class can have one or two C8-C18 alkyl or aralkyl chains and can be represented by the following formula:

R¹R²NR³R⁴⁺X⁻.

where R¹ and R² are C1-C18 linear or branched alkyl, alkaryl chains can be substituted in available positions by N, O, or S provided at least one R¹ or R² is a C8-C18 linear or branched alkyl chains can be substituted in available positions by N, O, or S. R³ and R⁴ are C1-C6 alkyl groups. R³ and R⁴ can also form a ring (e.g., a pyridine ring with the nitrogen of the quaternary ammonium group). X can be an anion, e.g., a halide, and can be Cl⁻ or Br⁻. Other anions can include methosulfate, ethosulfate, and phosphates. Compounds of this class include monoalkyltrimethyl ammonium salts, monoalkyldimethylbenzyl ammonium salts, dialkyldimethyl ammonium salts, benzethonium chloride, and octenidine.

Referring again to FIG. 1, in one embodiment, the shell 102 can be made by forming tooth or teeth-retaining cavities in a polymeric film including the antimicrobial compound or mixture of compounds. In some embodiments, the film can be easily and cost-effectively made from a reactive mixture prepared in an extruder. Examples of suitable extruders include a twin-screw extruder or a planetary extruder. Suitable twin-screw extruders include a co-rotating twin-screw extruder or a counter-rotating twin-screw extruder.

The components of the reactive mixture such as the base polymer for the shell 102 and the antimicrobial compound or mixture of compounds (e.g., monolaurin and PETg) can be individually or simultaneously fed into the extruder.

Through extrusion, a stream of molten polymeric material is formed and extruded through a die to form a uniform layer. An example of a suitable die includes a coat hanger die. The uniform layer can optionally be further pressed by a cold roller which thermally quenches the reaction shaping the polymeric material, thereby solidifying the polymeric material to obtain a sheet to be formed into an orthodontic appliance, such as the orthodontic appliance 100.

Multiple streams of molten polymeric material may be formed with multiple twin-screw extruders. The extrudable material in the streams may contain monolaurin and/or additional antimicrobial compounds. The multiple streams can be combined within a feed block as part of the process of forming the multi-layer sheet. The streams may comprise substantially the same material or may have substantially different material. For example, only some of the streams may contain the antimicrobial compounds such as monolaurin.

In some examples, the process may include melt blending extrudable material via three twin-screw extruders to form three separate streams of extrudable material. The three separate streams can be combined within a feed block to a die to form the sheet into a multi-layer sheet. One or more of the streams may contain monolaurin and/or additional antimicrobial compounds. For example, a first and second stream may be substantially similar and can optionally include monolaurin.

When combining multiple streams, the streams can be applied, continuously or discontinuously, in at least one of a down-stream direction, a cross-stream direction, and a combination thereof. For example, continuous streams with antimicrobial compounds, such as monolaurin, can be combined on either side of a discontinuous stream with no antimicrobial compounds.

The extrusion can occur at any suitable temperature. For example, the temperature can be in a range of from about 40° C. to about 230° C., or about 90° C. to about 200° C. The extrusion can occur for any suitable amount of time. For example, the extrusion can occur for a period of time ranging from about 0.5 hours to about 17 hours, or about 1 hour to about 6 hours.

In some embodiments, the neat form of an antimicrobial compounds such as antimicrobial lipids (e.g., monolaurin) can be injected into a molten polymer stream to form a blend just prior to extrusion into the desired shape. After extrusion, an annealing step can be carried out to enhance hydrophilicity. Annealing can occur at a temperature and for a time sufficient to increase the amount of the antimicrobial compound at the surface of the dental appliance.

In some embodiments, prior to extrusion, the antimicrobial compounds such as antimicrobial lipids (e.g., monolaurin) can be blended or otherwise uniformly mixed with pelletized or powdered polymer and subsequently melt extruding.

A liquid composition including at least one antimicrobial compound can be applied by, e.g., dipping, spraying, padding, or by brush or sponge, to form a composition layer on at least a portion of the entire exterior surface of the shell 102. In some embodiments, the method for applying the liquid composition includes, before thermoforming, spraying the liquid composition on the polymeric material. In some embodiments, the liquid composition can be applied after the shell 102 is thermoformed to have cavities 104 that are configured to receive one or more teeth. Also, the liquid composition can be applied to the shell before and after thermoforming, and the shell 102 can be partially or completely coated with the liquid composition.

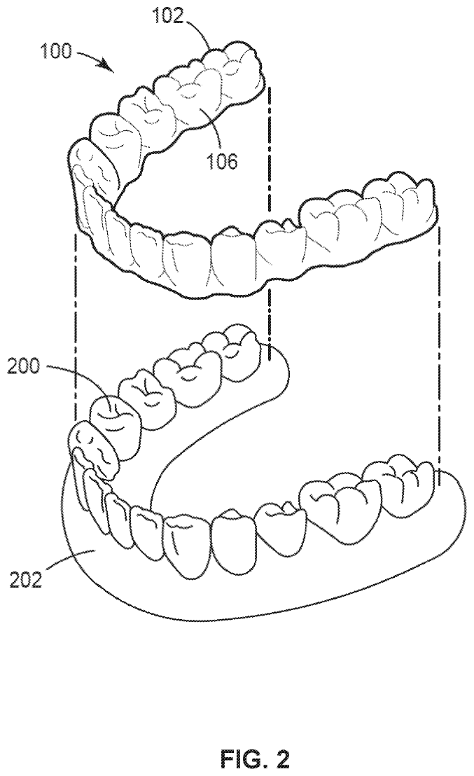

Referring now to FIG. 2, the shell 102 of the orthodontic appliance 100 is an elastic polymeric material that generally conforms to a patient's teeth 200, but that is slightly out of alignment with the patient's initial tooth configuration. In some embodiments, the shell 102 may be one of a group or a series of shells having substantially the same shape or mold, but which are formed from different materials to provide a different stiffness or resilience as need to move the teeth of the patient. In this manner, in one embodiment, a patient or a user may alternately use one of the orthodontic appliances during each treatment stage depending upon the patient's preferred usage time or desired treatment time period for each treatment stage.

No wires or other means may be provided for holding the shell 102 over the teeth 200, but in some embodiments, it may be desirable or necessary to provide individual anchors on teeth with corresponding receptacles or apertures in the shell 102 so that the shell 102 can apply a retentive or other directional orthodontic force on the tooth which would not be possible in the absence of such an anchor.

The shell 102 may be customized, for example, for daytime use and nighttime use, during function or non-function (chewing vs. non-chewing), during social settings (where appearance may be more important) and non-social settings (where the aesthetic appearance may not be a significant factor), or based on the patient's desire to accelerate the teeth movement (by optionally using the more stiff appliance for a longer period of time as opposed to the less stiff appliance for each treatment stage).

For example, in one aspect, the patient may be provided with a clear orthodontic appliance that may be primarily used to retain the position of the teeth, and an opaque orthodontic appliance that may be primarily used to move the teeth for each treatment stage. Accordingly, during the daytime, in social settings, or otherwise in an environment where the patient is more acutely aware of the physical appearance, the patient may use the clear appliance. Moreover, during the evening or nighttime, in non-social settings, or otherwise when in an environment where physical appearance is less important, the patient may use the opaque appliance that is configured to apply a different amount of force or otherwise has a stiffer configuration to accelerate the teeth movement during each treatment stage. This approach may be repeated so that each of the pair of appliances are alternately used during each treatment stage.

Referring to FIG. 2, systems and method in accordance with the various embodiments of the of the techniques of this disclosure include a plurality of incremental position adjustment appliances, each formed from the same or a different material, for each treatment stage of orthodontic treatment. The orthodontic appliances may be configured to incrementally reposition individual teeth 200 in an upper or lower jaw 202 of a patient. In some embodiments, the cavities 104 are configured such that selected teeth will be repositioned, while others of the teeth will be designated as a base or anchor region for holding the repositioning appliance in place as it applies the resilient repositioning force against the tooth or teeth intended to be repositioned.

Placement of the elastic shell 102 over the teeth 200 applies controlled forces in specific locations to gradually move the teeth into the new configuration. Repetition of this process with successive appliances having different configurations eventually moves a patient's teeth through a series of intermediate configurations to a final desired configuration.

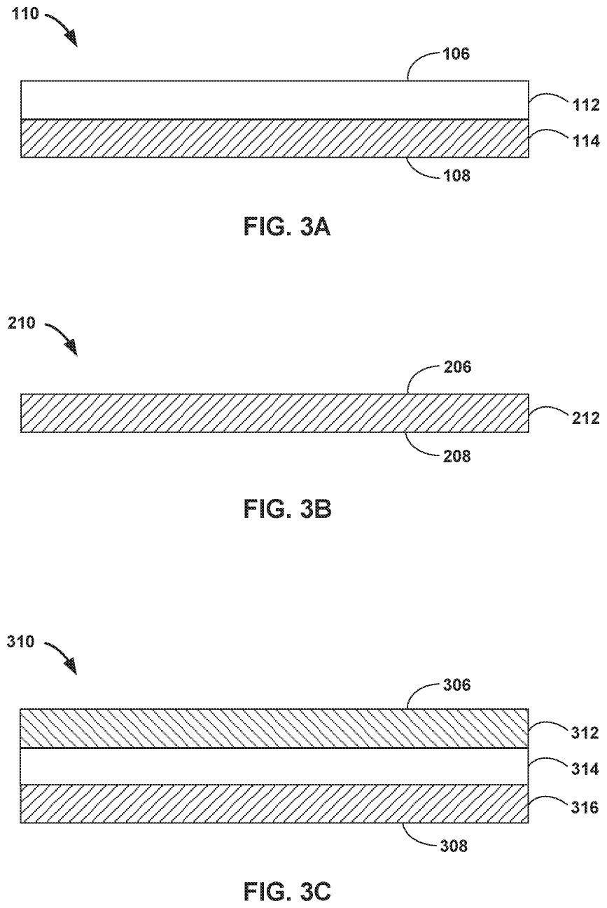

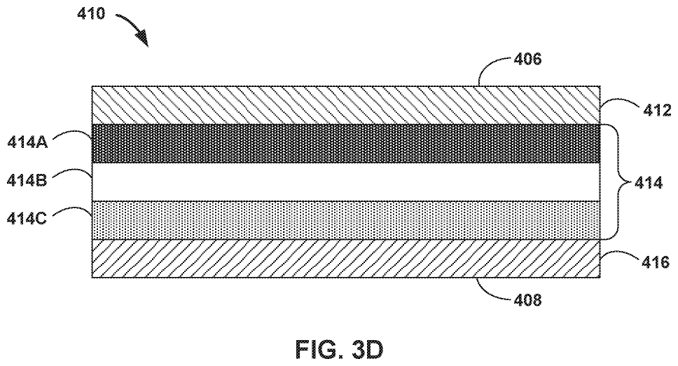

As shown in FIG. 3A, a multilayered polymeric film construction that may be formed into the dental appliance 100 (FIG. 1) having two layers, an outer layer 112 and an inner layer 114. The outer layer 112 can be thermoformed to form the first major external surface 106 of the shell 102 (FIG. 1), and the inner layer 114 may then form the second major internal surface 108 of the shell 102. In some examples, only the inner layer 114, or a combination of the outer layer 112 and the inner layer 114 have an antimicrobial compound or mixture of compounds such as monolaurin blended therein.

In some examples, when the shell 102 of the dental appliance has a multilayer construction, each layer can have an antimicrobial compound or mixture of compounds evenly blended throughout, have an antimicrobial compound concentration gradient, have no antimicrobial compound, or combinations thereof. For example, when the shell 102 has a layer construction 110 including four layers, there can be one layer with no antimicrobial compounds, one layer with an antimicrobial compound such as monolaurin blended evenly throughout, another layer with an antimicrobial compound such as monolaurin in a concentration gradient (i.e., monolaurin increasing consistently from one surface to another surface of the layer), or any combination thereof.

Generally, antimicrobial effect is achieved in body fluids such as saliva, plasma, serum or urine at concentrations less than 10 ppm. In some embodiments, Ag⁺ release concentration from the article can be 0.1 ppm, 0.5 ppm, 1 ppm, 2 ppm, 2.5 ppm, 3 ppm, 4 ppm, 5 ppm, 6 ppm, 7 ppm, 8 ppm, 9 ppm, 10 ppm, 20 ppm, 40 ppm or a range between and including any two of these values.

In some embodiments, the layer of the shell 102 with monolaurin can have less than a certain wt % monolaurin, including 0.1 wt %, 0.5 wt %, 1 wt %, 2 wt %, 2.5 wt %, 3 wt %, 4 wt %, 5 wt %, 6 wt %, 7 wt %, 8 wt %, 9 wt %, 10 wt %, 20 wt %, 40 wt % or a range between and including any two of these values (e.g., between 1 wt % and 3 wt % monolaurin or between 0.7 wt % and 7.5 wt %). In some embodiments, the layer of the shell 102 can have an effective amount of monolaurin to exhibit at least a selected log reduction of at least one of S. mutans, S. aureus, P. aeruginosa, and combinations thereof, following 24-hour contact. In some embodiments, the log reduction can be at least 0.1, 0.5, 1, 2, 2.5, 3, 4, 5, 6, 7, 8, 9, 10 or a range between and including any two of these values.

An antimicrobial compound such as monolaurin can have varying concentration gradients, including as described in the examples below. For example, a monolaurin concentration gradient can increase or decrease, continuously or discontinuously, from one surface to another surface. The monolaurin concentration gradient can also increase or decrease, continuously or discontinuously, from the middle of the layer to the surface of the layer.

In some examples, the shell 102 can have one or more layers of the layer construction 110 applied in various patterns, e.g., continuous or discontinuous structures or strips of material. For example, the layer construction 110 can include a layer of a polymeric material including monolaurin applied in discontinuous strips on a layer with no monolaurin, or a layer with no monolaurin applied in discontinuous strips between two layers with monolaurin blended throughout. The strips or structures of the layers of the shell 102 can have varying shapes and sizes arranged in uniform or non-uniform patterns. The various shapes include but are not limited to triangular, circular, lenticular, elliptical, conical, irregular, and combinations thereof.

In some embodiments, the shell 102 can include dyes or pigments to provide a desired color that may be, for example, decorative or selected to improve the appearance of the teeth of the patient.

In various embodiments, the shell 102 can have at least one of an antimicrobial, an antibacterial, or an anti-biofilm, effect. In addition to the monolaurin, the shell 102 can include a wide variety of antimicrobial compounds, as long as the shell 110 exhibits at least a 1-log microbial reduction against S. aureus and S. mutans following 24-hour contact. In some embodiments, the shell 102 has at least a 2-log microbial reduction against S. aureus and S. mutans following 24-hour contact. In some embodiments, the shell 102 has at least a 3-log microbial reduction against S. aureus and S. mutans following 24-hour contact.

Log reductions are measured after testing according to ISO test method ISO 22196:2011, “Measurement of antibacterial activity on plastics and other non-porous surfaces,” with appropriate modifications of the test method to accommodate the test materials as described below.

As shown in FIG. 3B, a layer construction 210 can be a single layer 212 and can be similar to the layer construction 110 except for the differences described herein. For example, with the single layer 212 includes monolaurin and may include additional antimicrobial compounds. The single layer 212 includes a first major external surface 206 and a second major internal surface 208 that contacts the teeth of the patient. In some examples, the layer construction 210 includes a 30 thousandths of an inch (mil) (0.762 millimeter (mm)) layer including monolaurin with at least one of Tritan, PETg, PCTg, Neostar Elastomer FN007, or TPU.

As shown in FIG. 3C, a layer construction 310 can include an outer layer 312, a core layer 314, and an inner layer 316. The layer construction 310 can be similar to the layer construction 110 except for the differences described herein. The outer layer 312 includes a first major external surface 306 of the shell 102, and the inner layer 316 includes a second major internal surface 308 of the shell 102. The outer layer 312 and inner layer 316 can include monolaurin and the core layer can be substantially free of monolaurin. In one example, the outer layer 312 and the inner layer 316 can include monolaurin with Tritan, PCTg, or PETg; and the core layer 314 can include Neostar Elastomer FN007, Elvaloy resins, Admer adhesive resins, TPU, or Versaflex TPE. In another example, the outer layer 312 and the inner layer 316 can include monolaurin with Neostar Elastomer FN007, Elvaloy resins, Admer adhesive resins, or TPU; and the core layer 314 can include Tritan, PCTg, or PETg. The outer layer 312, the core layer 314, and the inner layer 316 can have varying dimensions include varying thicknesses of between 0.5 mil and 25 mil, 1 mil and 18 mil, 5 mil and 15 mil, 7 mil and 12 mil, and can include 1 mil, 5 mil, 10 mil and 18 mil. The thickness of the layer construction 310 can include varying thicknesses of between 15 mil and 40 mil, 20 mil and 35 mil, and can include 30 mil.

In some examples, as shown in FIG. 3D, the shell 102 can have a layer construction

410 with an outer layer 412, a core 414, and an inner layer 416. The layer construction 410 can be similar to the layer construction 110 except for the differences described herein. The core 414 can have one or more layers. For example, the core 414 can have an outer core layer 414A, a middle core layer 414B, and an inner core layer 414C. The outer layer 412 includes a first major external surface 406 of the shell 102, and the inner layer 416 includes a second major internal surface 408 of the shell 102 that contacts the surface of the teeth.

In some examples, only two layers of the shell 102 can have monolaurin blended therein: the outer layer 412 and the inner layer 416. The number of layers for the core 414 can vary. For example, the core 414 can have only one layer similar to the example shown in FIG. 3C. The core can also have more than three layers. Depending on the materials of the shell 102 and the performance of the shell 102, the core 414 can have as many layers as needed. In one example, the outer layer 412 and the inner layer 416 can include monolaurin with Tritan, PCTg, or PETg; the outer core layer 414A and the inner core layer 414C can include Neostar Elastomer FN007, Elvaloy resins, Admer adhesive resins, or TPU; and the middle core layer 414B can include Tritan, PCTg, or PETg. The outer layer 312, the core layer 314, and the inner layer 316 can have varying dimensions include varying thicknesses of between 0.5 mil and 25 mil, 1 mil and 18 mil, 5 mil and 15 mil, 7 mil and 12 mil, and can include 1 mil, 5 mil, 10 mil and 18 mil. The thickness of the layer construction 410 can include varying thicknesses of between 15 mil and 40 mil, 20 mil and 35 mil, and can include 30 mil.

The devices of the present disclosure will now be further described in the following non-limiting examples.

### EXAMPLES

Summary of Test Procedures

Deflection Test

The deflection test is performed according to the test method generally described in Paragraphs 0023-0025 of US Publication No. 2013/0078593 (Andreiko) in both dry and wet conditions. Film samples were cut into 0.5 inch (12.5 mm) wide strips and woven between 5 colinear, 0.125 inch (3.175 mm) diameter pegs, with a center-to-center spacing of 0.67 inches (17 mm). As a result, the samples take on a wave-like or sinusoidal shape. The deflection of a sample is defined as the amplitude of this wave, i.e., the distance between the outside surface of the center peak and a line drawn between the outside surfaces of the neighboring peaks. When installed on the pegboard, assuming the film strips hug the pegs tightly, the deflection is ideally equal to the peg diameter plus twice the sample thickness, or 0.185 inches (4.7 mm). In reality, the samples may not follow the peg surface contours perfectly, and the peak-to-peak deflection may be slightly more than 0.185 inches (4.7 mm).

Samples were installed in pegboard holders and held in place for 7 days at 37° C. in an oven. For the dry tests, the samples were exposed to oven air. For the wet tests, the samples were immersed in water for the entire 7 days in the oven. After 7 days, the samples were removed from the pegboards, and the deflection was measured to quantify the degree of plastic deformation.

For a wet-dry deflection test, half of the samples are kept dry and half immersed during time in the oven. The wet-dry result is the difference between the average deflection measured in the wet samples and the average deflection measured in the dry samples. A lower value can be advantageous as it may indicate the impact from hydration by water is reduced or minimized.

Haze, Transmission and Clarity

Haze, transmission and clarity were determined using a HAZE-GARD PLUS meter available from BYK-Gardner Inc., Silver Springs, Md., which was designed to comply with the ASTM D1003-13 standard. The specimen surface is illuminated perpendicularly with the transmitted light, measured with an integrating sphere (0°/diffuse geometry). The spectral sensitivity conforms to CIE standard spectral value function “Y” under illuminant C with a 2° observer.

Coefficient of Friction (COF)

Static and dynamic coefficients of friction were measured according to ASTM D1894. All measurements were made in the machine direction—no transverse measurements were performed. Dynamic coefficient of friction calculations were based on the load data gathered between 20 and 40 mm of displacement.

Log Reduction Testing Method

A modified ISO 22196:2011, “Measurement of antibacterial activity on plastics and other non-porous surfaces” was used to evaluate the antibacterial activity of coatings. The bacterial inoculum was prepared in a Butterfield's Buffer at concentration of E8 CFU/ml. A portion of the bacterial suspension (1 ml) was placed into 9.0 mL of artificial saliva (prepared according to Table 1 below). Then, a portion of bacteria suspension in artificial saliva (150 ul) placed onto the surface of the article (1 sq. inch) and the inoculated article was incubated for the specified contact time at 37+/−1° C. After incubation, the article was placed into 45 ml of D/E Neutralizing Broth (available from Sigma-Aldrich Corp., St. Louis, Mo.). The number of surviving bacteria in the Neutralizing Broth was determined by using 3M PETRIFILM, available from 3M Company, Saint Paul, Minn.

TABLE 1
Composition of Artificial Saliva:

| | | | DI | |
|---|---|---|---|---|
| | | Gastric | water | 400 mL |
| 2.2 | g/L | Mucin | 100% | 0.880 |
| 0.381 | g/L | NaCl | 100% | 0.152 |
| 0.213 | g/L | CaCl₂•2H₂O | 100% | 0.085 |
| 0.738 | g/L | KH₂PO₄ | 99.8% | 0.296 |
| 1.114 | g/L | KCl | 99.8% | 0.446 |
| 400 | mL | DI Water | | |

Mix well. Adjust pH to 7.0 with NaOH solution.

### Comparative Example 1

30 thousandths of an inch (mil) (0.762 millimeter (mm)) PETg sheet was obtained from Pacur, Oshkosh, Wis. PETg sheet was used as a single layer sheet.

### Example 1

30 thousandths of an inch (mil) (0.762 millimeter (mm)) trilayer sheet was made by coextrusion using three twin-screw extruders. The core layer of the trilayer sheet was a 10 mil (0.254 mm) 100% PETg layer (Eastar GN071, available from Eastman Chemical, Kingsport, Tenn.) and the skin layers (the interior layer and exterior layer) were a 10 mil (0.254 mm) layer of 2 wt % monolaurin and 98 wt % PETg. The core layer was located between the skin layers. Three melt streams were extruded from three twin-screw extruders via a feed block to an 18 inch (45.72 centimeters) single layer die. The diameters of the three extruders were 25 millimeters (mm) (1^{st} skin layer), 40 mm (core layer) and 25 mm (2^{nd} skin layer), respectively. Monolaurin was dosed into 25 mm extruders hoppers by gravimetric powder feeders. The melt temperature was operated at approximately 480 degrees Fahrenheit (° F.) (249 degrees Celsius (° C.)).

Table 2 displays the differences between the films of Comparative Example 1 and the Example 1.

TABLE 2
Comparison of PETg sheet (Comparative Example 1)
to a Trilayer Sheet with Monolaurin (Example 1)

| | Comparative | |
|---|---|---|
| Properties | Example 1 | Example 1 |
| Transmission | 90.80% | 92.30% |
| Haze | 1% | 5.10% |
| Clarity | 99.60% | 89.20% |
| 24-hour log reduction against S. mutans | 0 | 3 |
| Coefficient of Friction (static) | 0.381 | 0.267 |
| Coefficient of Friction (dynamic) | 0.322 | 0.24 |
| Pegboard dry deflection after seven- days | 3.66 mm | 4.91 mm |
| aging at 37 degrees Celsius (° C.) | | |
| Pegboard wet deflection after seven- days | 5.2 mm | 4.91 mm |
| aging at 37 degrees Celsius (° C.) | | |
| Pegboard (wet-dry) deflection after seven- | 1.54 mm | 0 mm |
| days aging at 37 degrees Celsius (° C.) | | |

Both the films of Comparative Example 1 (90.80%) and the Example 1 (92.30%) had a visible light transmission greater than 85%. Both the films of Comparative Example 1 (1%) and the Example 1 (5.10%) had a haze level of less than 20%. Both the films of Comparative Example 1 (99.60%) and the Example 1 (89.20%) displayed a clarity greater than 75%.

For 24-hour log reduction against S. mutans, the film of Comparative Example 1 displayed a zero-log reduction and Example 1 displayed a 3-log reduction. A 2-log or greater reduction can be considered to be effective in odor reduction. The film of Comparative Example 1 displayed an ineffective odor reduction, and the film of Example 1 displayed an effective odor reduction.

The film of Comparative Example 1 displayed a coefficient of friction (static) of 0.381, and the film of Example 1 displayed a coefficient of friction (static) of 0.267. Thus, the film of Example 1 displayed an approximately 30% reduction coefficient of friction (static) from the film of Comparative Example 1.

The film of Comparative Example 1 displayed a coefficient of friction (dynamic) of 0.322, and the film of Example 1 displayed a coefficient of friction (dynamic) of 0.240. Thus, the film of example 1 displayed approximately 25% reduction coefficient of friction (dynamic) compared to the film of Comparative Example 1.

For the film of Comparative Example 1, there was a 1.54 mm difference in pegboard (wet-dry) deflection after 7 days. For the film of Example 1, there was no difference in pegboard (wet-dry) deflection after 7 days. A lower value can be preferred for the difference in pegboard (wet-dry) deflection after 7 days because this indicates the impact from hydration by water is minimized.

Various embodiments have been described. These and other embodiments are within the scope of the following claims.

1. A dental appliance comprising:
a polymeric shell with an arrangement of one or more cavities configured to receive one or more teeth, wherein the polymeric shell comprises an antimicrobial lipid.

2. The dental appliance of claim 1, wherein the antimicrobial lipid comprises monolaurin.

3. The dental appliance of claim 1, wherein the antimicrobial lipid comprises an effective amount of monolaurin to reduce at least one of S. mutans, S. aureus, P. aeruginosa, and combinations thereof, following 24-hour contact.

4. The dental appliance of claim 1, wherein the antimicrobial lipid comprises between 1 weight percent (wt %) and 3 wt % monolaurin.

5. The dental appliance of claim 1, wherein the polymeric shell comprises an outer layer with a first major external surface, an inner layer with a second major internal surface configured to contact the one or more teeth, and a core layer between the outer layer and the inner layer.

6. The dental appliance of claim 5, wherein the inner layer and the outer layer comprise the antimicrobial lipid, and wherein the core layer is substantially free of the antimicrobial lipid.

7. The dental appliance of claim 1, wherein the antimicrobial lipid is a first antimicrobial lipid compound and wherein the polymeric shell comprises at least a second antimicrobial lipid compound.

8. The dental appliance of claim 7, wherein the second antimicrobial lipid compound comprises at least one of a metal, metal oxide, cationic surfactant free of aromatic groups, cationic antimicrobial polymer, antimicrobial lipid, alkyl carboxylic acid, an alkyl carboxylate ester carboxylic acid, and mixtures and combinations thereof, and wherein the cationic antimicrobial polymer is at least one of a polybiguanide and a polyquaternary amine.

9. (canceled)

10. The dental appliance of claim 1, wherein the polymeric shell comprises a polymer chosen from polyamide, polyethylene terephthalate, polybutylene terephthalate, polyester/polycarbonate copolymer, polyolefin, cyclic olefin polymer, styrenic copolymer, polyetherimide, polyetheretherketone, polyethersulfone, polytrimethylene terephthalate, parylene and mixtures and combinations thereof.

11. The dental appliance of claim 1, wherein the polymeric shell is configured to provide resistance to an impact from hydration by water for enhancing force persistence of the dental appliance.

12. The dental appliance of claim 1, wherein after aging seven-days at 37 degrees Celsius (° C.), a deflection of the dental appliance is substantially similar for either a wet dental appliance according to the Wet Deflection Test or a dry dental appliance according to the Dry Deflection Test.

13-14. (canceled)

15. A method comprising:
melt blending an antimicrobial lipid with a first extrudable material to form a stream;
extruding the stream to form a sheet; and
shaping the sheet to form an arrangement of one or more cavities configured to receive one or more teeth.

16. The method of claim 15, wherein extruding the stream to form the sheet comprises extruding the stream via a feed block to a die.

17. The method of claim 15, wherein the stream is a first stream and melt blending the antimicrobial lipid with the first extrudable material comprises blending with a first twin-screw extruder, further comprising melt blending a second extrudable material to form a second stream via a second twin-screw extruder.

18. The method of claim 17, wherein the first stream and the second stream are combined to form the sheet and the first stream is applied discontinuously in at least one of a down-stream direction, a cross-stream direction, and a combination thereof.

19. A method comprising:
forming a polymeric shell comprising a plurality of cavities in a first major surface thereof, wherein the polymeric shell comprises an antimicrobial lipid, and
wherein the cavities are configured to receive one or more teeth.

20. The method of claim 19, wherein forming the polymeric shell comprises extruding a polymeric material to form a sheet of polymeric material and the sheet of polymeric material is formed into the polymeric shell.

21. The method of claim 19, wherein the sheet of polymeric material is coated with a composition layer comprising the antimicrobial lipid prior to the sheet of polymeric material being formed into a polymeric shell.

22. The method of claim 19, further comprising, after forming the polymeric shell, applying a coating composition layer to the formed polymeric shell, wherein the coating composition layer comprises the antimicrobial lipid.

23. The method of claim 19, wherein the antimicrobial lipid is monolaurin.
